(19) United States
(12) Patent Application Publication
(10) Pub. No.: US 2004/0132210 A1
(43) Pub. Date: Jul. 8, 2004

(54) Assay method using a biochemical analysis unit and biochemical analysis apparatus

(75) Inventor: Kenji Nakajima, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/692,011

(22) Filed: Oct. 24, 2003

(30) Foreign Application Priority Data
Oct. 24, 2002 (JP) 309686/2002

(51) Int. Cl. G01N 33/558

(52) U.S. Cl. 436/514

(57) ABSTRACT

A specific binding detecting process is performed, wherein a receptor or a ligand is forcibly caused to flow such that the receptor or the ligand flows across each of porous adsorptive regions of a biochemical analysis unit, to which ligands or receptors have been bound, the receptor or the ligand being specifically bound to at least one of the ligands or the receptors, and wherein the receptor or the ligand having thus been bound is detected by the utilization of a labeling substance. During the process, a liquid is forcibly caused to flow, such that the liquid flows across each of the porous adsorptive regions. A liquid having been subjected to gas content decreasing processing is employed as the liquid, which is forcibly caused to flow.

### BACKGROUND OF THE INVENTION

1. Field of the Invention

This invention relates to an assay method for detecting a receptor or a ligand. This invention particularly relates to an assay method for detecting a receptor or a ligand by the utilization of a biochemical analysis unit provided with porous adsorptive regions. This invention also relates to a biochemical analysis apparatus for carrying out the assay method.

2. Description of the Related Art

Various biochemical analysis systems have heretofore been used. With the biochemical analysis systems, for example, ligands or receptors (i.e., the substances, which are capable of specifically binding to organism-originating substances and whose base sequences, base lengths, compositions, characteristics, and the like, are known) are spotted with a spotter device onto different positions on a surface of a supporting material of a glass array, which utilizes a slide glass plate, or the like, or a membrane array, which utilizes a membrane filter, or the like. The spotted ligands or receptors are then fixed to adsorptive regions of the supporting material. Examples of the thus spotted ligands or the thus spotted receptors include hormones, tumor markers, enzymes, antibodies, antigens, abzymes, other proteins, nucleic acids, cDNA's, DNA's, and RNA's. Thereafter, a labeled receptor or a labeled ligand, which has been labeled with a fluorescent labeling substance, such as a fluorescent substance or a fluoro chrome, is subjected to hybridization, or the like, with the ligands or the receptors, which have been fixed to the adsorptive regions of the supporting material. The labeled receptor or the labeled ligand is thus specifically bound to at least one of the ligands or the receptors, which have been fixed to the adsorptive regions of the supporting material. The labeled receptor or the labeled ligand is the substance, which has been sampled from an organism through extraction, isolation, or the like, or has been subjected to chemical treatment after being sampled, and which has been labeled with the fluorescent labeling substance. Examples of the labeled receptors or the labeled ligands include hormones, tumor markers, enzymes, antibodies, antigens, abzymes, other proteins, nucleic acids, DNA's, and mRNA's. Thereafter, excitation light is irradiated to the adsorptive regions of the supporting material, and the fluorescent labeling substance, which is contained selectively in the adsorptive region of the supporting material, is excited by the excitation light to produce fluorescence. The thus produced fluorescence is detected photoelectrically. In accordance with the results of the detection of the fluorescence, the organism-originating substance is analyzed.

With the biochemical analysis systems described above, a large number of the ligands or the receptors are formed at a high density at different positions on the surface of the supporting material of the membrane filter, or the like, and the labeled receptor or the labeled ligand, which has been labeled with the fluorescent labeling substance, is subjected to the hybridization, or the like, with the ligands or the receptors, which have been formed at a high density at different positions on the surface of the supporting material. Therefore, the biochemical analysis systems described above have the advantages in that an organism-originating substance is capable of being analyzed quickly.

The biochemical analysis systems described above are required to enable the detection with a sufficiently high accuracy, an enhanced detection limit, and enhanced reproducibility. However, with the biochemical analysis systems utilizing the fluorescent labeling substance, since the detection sensitivity is low, it is necessary that a large amount of the labeled receptor or a large amount of the labeled ligand be utilized for the expression analysis. Also, with the biochemical analysis systems utilizing the fluorescent labeling substance, the problems occur in that, for example, the amount of each of the ligands or the receptors capable of being fixed to the glass array is small, and that the ligands or the receptors having been fixed to the glass array peel off from the glass array during the processes of the analysis operation.

[Patent literature 1] U.S. Pat. No. 5,543,295

[Non-patent literature 1] “Nature Genetics,” Vol. 21, pp. 25-32, 1999

[Non-patent literature 2] “Bioindustry,” Vol. 18, pp. 13-19, 2001

Heretofore, with the biochemical analysis systems described above, the hybridization, or the like, has ordinarily been performed with a shaking technique. With the shaking technique, the experimenter manually puts an array, on which the ligands or the receptors have been fixed, into a hybridization bag and adds a reaction liquid, which contains the labeled receptor or the labeled ligand, into the hybridization bag. Also, the experimenter manually gives vibrations to the hybridization bag, and the labeled receptor or the labeled ligand is thus moved through convection or diffusion. In this manner, the labeled receptor or the labeled ligand is specifically bound to one of the ligands or the receptors having been fixed on the array.

However, with the shaking technique described above, it is not always possible to achieve efficient diffusion of the labeled receptor or the labeled ligand, which is contained in the hybridization reaction liquid, through each of the plurality of the adsorptive regions, which contain the ligands or the receptors. Therefore, the problems occur in that the ligands or the receptors and the labeled receptor or the labeled ligand cannot efficiently be subjected to the hybridization. In cases where the labeled receptor or the labeled ligand, which is contained in the hybridization reaction liquid, cannot be sufficiently diffused through each of the plurality of the adsorptive regions, which contain the ligands or the receptors, a ratio of the intensity of the emitted light (signal), which intensity corresponds to the amount of the labeled receptor or the labeled ligand having been bound to the adsorptive region, to the intensity of the emitted light (noise or background) of an adsorptive region, to which the labeled receptor or the labeled ligand has not been bound, cannot be kept high. (The signal-to-noise ratio of the signal representing the intensity of the emitted light, which intensity corresponds to the amount of the labeled receptor or the labeled ligand having been bound to the adsorptive region, to the noise or the background cannot be kept high.) Accordingly, in cases where the amount of the labeled receptor or the labeled ligand, which is bound to the adsorptive region, is small, it becomes difficult for the labeled receptor or the labeled ligand to be detected.

It may be considered that, in order for the labeled receptor or the labeled ligand to penetrate sufficiently into the interior of each of the adsorptive regions, the reaction liquid may be forcibly circulated through the interior of each of the adsorptive regions. However, in cases where the reaction liquid is pressurized such that the reaction liquid may be forcibly circulated through the interior of each of the adsorptive regions, the pressure exerted to the reaction liquid becomes low after the reaction liquid has passed through each of the adsorptive regions, and bubbles arise due to cavitation. The bubbles having thus been formed cling to the surfaces of the adsorptive regions and cause the flow of the reaction liquid to be biased. Therefore, the problems occur in that the signal-to-noise ratio becomes low, and the signal-to-noise varies for different positions of the adsorptive regions. Also, the problems often occur in that the bubbles clinging to the surfaces of the adsorptive regions obstruct the detection of the labeled receptor or the labeled ligand.

### SUMMARY OF THE INVENTION

The primary object of the present invention is to provide an assay method using a biochemical analysis unit, wherein problems are capable of being prevented from occurring in that, in cases where a reaction liquid is forcibly circulated through the interior of each of adsorptive regions of the biochemical analysis unit, a signal-to-noise ratio becomes low, and the signal-to-noise varies for different positions of the adsorptive regions.

Another object of the present invention is to provide a biochemical analysis apparatus for carrying out the assay method using a biochemical analysis unit.

The present invention provides a first assay method using a biochemical analysis unit, comprising the steps of:

i) obtaining a biochemical analysis unit provided with a plurality of porous adsorptive regions, to which ligands or receptors have been bound respectively, and

ii) performing a specific binding detecting process comprising the steps of:

a) forcibly causing a receptor or a ligand to flow such that the receptor or the ligand flows across each of the porous adsorptive regions of the biochemical analysis unit, the receptor or the ligand being thus subjected to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and

b) detecting the receptor or the ligand, which has thus been specifically bound to at least one of the ligands or at least one of the receptors, by the utilization of a labeling substance,

a liquid being forcibly caused to flow, such that the liquid flows across each of the porous adsorptive regions of the biochemical analysis unit, during the specific binding detecting process,

wherein a liquid, which has been subjected to gas content decreasing processing for decreasing the content of a dissolved gas, is employed as the liquid, which is forcibly caused to flow.

In the first assay method using a biochemical analysis unit in accordance with the present invention, all of liquids, which are forcibly caused to flow, may be the liquids having been subjected to the gas content decreasing processing for decreasing the content of the dissolved gas. Alternatively, for example, only the liquid, which contains the receptor or the ligand, may be the liquid having been subjected to the gas content decreasing processing for decreasing the content of the dissolved gas.

The present invention also provides a second assay method using a biochemical analysis unit, comprising the steps of:

i) obtaining a biochemical analysis unit provided with a plurality of porous adsorptive regions, to which ligands or receptors have been bound respectively, and

ii) performing a specific binding detecting process comprising the steps of:

a) forcibly causing a receptor or a ligand to flow such that the receptor or the ligand flows across each of the porous adsorptive regions of the biochemical analysis unit, the receptor or the ligand being thus subjected to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and

b) detecting the receptor or the ligand, which has thus been specifically bound to at least one of the ligands or at least one of the receptors, by the utilization of a labeling substance,

a liquid being forcibly caused to flow, such that the liquid flows across each of the porous adsorptive regions of the biochemical analysis unit, during the specific binding detecting process,

wherein bubble removing processing for removing bubbles, which are present in the liquid, from the liquid is performed during the flowing of the liquid.

In the second assay method using a biochemical analysis unit in accordance with the present invention, a liquid, which has been subjected to the gas content decreasing processing for decreasing the content of the dissolved gas, may be employed as the liquid, which is forcibly caused to flow.

The present invention further provides a third assay method using a biochemical analysis unit, comprising the steps of:

i) obtaining a biochemical analysis unit provided with a plurality of porous adsorptive regions, to which ligands or receptors have been bound respectively, and

ii) performing a specific binding detecting process comprising the steps of:

a) forcibly causing a receptor or a ligand to flow such that the receptor or the ligand flows across each of the porous adsorptive regions of the biochemical analysis unit, the receptor or the ligand being thus subjected to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and

b) detecting the receptor or the ligand, which has thus been specifically bound to at least one of the ligands or at least one of the receptors, by the utilization of a labeling substance,

a liquid being forcibly caused to flow, such that the liquid flows across each of the porous adsorptive regions of the biochemical analysis unit, during the specific binding detecting process,

wherein bubble dissolving processing for dissolving bubbles, which are present in the liquid, is performed during the flowing of the liquid.

In the third assay method using a biochemical analysis unit in accordance with the present invention, a liquid, which has been subjected to the gas content decreasing processing for decreasing the content of the dissolved gas, may be employed as the liquid, which is forcibly caused to flow.

The first, second, and third assay methods using a biochemical analysis unit in accordance with the present invention may be modified such that the specific binding detecting process comprises the steps of:

a) forcibly causing a reaction liquid containing a labeled receptor or a labeled ligand, which has been labeled with a labeling substance, to flow such that the reaction liquid flows across each of the porous adsorptive regions of the biochemical analysis unit provided with the plurality of the porous adsorptive regions, to which the ligands or the receptors have been bound respectively, the labeled receptor or the labeled ligand being thus subjected to the specific binding with the ligands or the receptors, each of which has been bound to one of the porous adsorptive regions of the biochemical analysis unit, the labeled receptor or the labeled ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and

b) detecting the labeled receptor or the labeled ligand, which has thus been specifically bound to at least one of the ligands or at least one of the receptors, by the utilization of the labeling substance.

Also, the first, second, and third assay methods using a biochemical analysis unit in accordance with the present invention may be modified such that the specific binding detecting process comprises the steps of:

a) subjecting the receptor or the ligand to the specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,

b) forcibly causing a reaction liquid containing a labeled body, which has been labeled with a labeling substance, to flow such that the reaction liquid flows across each of the porous adsorptive regions of the biochemical analysis unit, the labeled body being thus specifically bound to the receptor or the ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and

c) detecting the receptor or the ligand, which has been specifically bound to at least one of the ligands or at least one of the receptors, by the utilization of the labeled body.

Further, the first, second, and third assay methods using a biochemical analysis unit in accordance with the present invention may be modified such that the specific binding detecting process comprises the steps of:

a) subjecting an auxiliary substance-bound receptor or an auxiliary substance-bound ligand, to which an auxiliary substance has been bound, to the specific binding with the ligands or the receptors, each of which has been bound to one of the porous adsorptive regions of the biochemical analysis unit, the auxiliary substance-bound receptor or the auxiliary substance-bound ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,

b) forcibly causing a reaction liquid containing a labeling substance, which is capable of undergoing specific binding with the auxiliary substance, to flow such that the reaction liquid flows across each of the porous adsorptive regions of the biochemical analysis unit, the labeling substance, which is capable of undergoing specific binding with the auxiliary substance, being thus specifically bound to the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and

c) detecting the auxiliary substance-bound receptor or the auxiliary substance-bound ligand, which has been specifically bound to at least one of the ligands or at least one of the receptors, by the utilization of the labeling substance.

The present invention still further provides a first biochemical analysis apparatus, comprising:

i) are action vessel, which is provided with a support section for releasably supporting a biochemical analysis unit within the reaction vessel, the biochemical analysis unit being provided with a plurality of porous adsorptive regions, to which ligands or receptors have been bound respectively, the reaction vessel being adapted to perform specific binding of a specific binding substance with the ligands or the receptors, each of which has been bound to one of the porous adsorptive regions of the biochemical analysis unit, the specific binding substance being capable of undergoing the specific binding with the ligands or the receptors, and

ii) flowing means for forcibly causing a reaction liquid containing the specific binding substance to flow within the reaction vessel such that the reaction liquid containing the specific binding substance flows across each of the porous adsorptive regions of the biochemical analysis unit,

wherein the apparatus further comprises bubble removing means for performing bubble removing processing for removing bubbles, which are present in the reaction liquid, from the reaction liquid, which is flowing.

The present invention also provides a second biochemical analysis apparatus, comprising:

i) a reaction vessel, which is provided with a support section for releasably supporting a biochemical analysis unit within the reaction vessel, the biochemical analysis unit being provided with a plurality of porous adsorptive regions, to which ligands or receptors have been bound respectively, the reaction vessel being adapted to perform specific binding of a specific binding substance with the ligands or the receptors, each of which has been bound to one of the porous adsorptive regions of the biochemical analysis unit, the specific binding substance being capable of undergoing the specific binding with the ligands or the receptors, and

ii) flowing means for forcibly causing a reaction liquid containing the specific binding substance to flow within the reaction vessel such that the reaction liquid containing the specific binding substance flows across each of the porous adsorptive regions of the biochemical analysis unit,

wherein the apparatus further comprises bubble dissolving means for performing bubble dissolving processing for dissolving bubbles, which are present in the liquid, on the reaction liquid, which is flowing.

With the first assay method using a biochemical analysis unit in accordance with the present invention, the biochemical analysis unit provided with the plurality of the porous adsorptive regions, to which the ligands or the receptors have been bound respectively, is obtained, and the specific binding detecting process is performed. The specific binding detecting process comprises the steps of: (a) forcibly causing the receptor or the ligand to flow such that the receptor or the ligand flows across each of the porous adsorptive regions of the biochemical analysis unit, the receptor or the ligand being thus subjected to the specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and (b) detecting the receptor or the ligand, which has thus been specifically bound to at least one of the ligands or at least one of the receptors, by the utilization of a labeling substance. During the specific binding detecting process, the liquid is forcibly caused to flow, such that the liquid flows across each of the porous adsorptive regions of the biochemical analysis unit. Also, the liquid, which has been subjected to the gas content decreasing processing for decreasing the content of the dissolved gas, is employed as the liquid, which is forcibly caused to flow. Therefore, with the first assay method using a biochemical analysis unit in accordance with the present invention, the occurrence of bubbles due to cavitation is capable of being suppressed. Accordingly, the problems are capable of being prevented from occurring in that the bubbles cling to the surfaces of the adsorptive regions and cause the flow of the liquid to be biased. As a result, the problems are capable of being prevented from occurring in that the signal-to-noise ratio becomes low, and in that the signal-to-noise varies for different positions of the adsorptive regions. Also, the problems are capable of being prevented from occurring in that the bubbles clinging to the surfaces of the adsorptive regions obstruct the detection of the receptor or the ligand.

With the second assay method using a biochemical analysis unit in accordance with the present invention, wherein the bubble removing processing for removing the bubbles, which are present in the liquid, from the liquid is performed during the flowing of the liquid, the same effects as those described above are capable of being obtained. Also, with the third assay method using a biochemical analysis unit in accordance with the present invention, wherein the bubble dissolving processing for dissolving the bubbles, which are present in the liquid, is performed during the flowing of the liquid, the same effects as those described above are capable of being obtained.

### BRIEF DESCRIPTION OF THE DRAWINGS

FIG. 1 is a schematic perspective view showing an example of a biochemical analysis unit utilized for the assay method using a biochemical analysis unit in accordance with the present invention,

FIG. 2 is a schematic view showing an example of a batch type of deaerator,

FIG. 3 is a schematic view showing an example of a continuous type of deaerator,

FIG. 4 is a schematic view showing an example of a reactor utilized for the assay method using a biochemical analysis unit in accordance with the present invention,

FIG. 5 is a schematic view showing an embodiment of the biochemical analysis apparatus in accordance with the present invention, and

FIG. 6 is a schematic view showing a different embodiment of the biochemical analysis apparatus in accordance with the present invention.

### DESCRIPTION OF THE PREFERRED EMBODIMENTS

The present invention will hereinbelow be described in further detail with reference to the accompanying drawings.

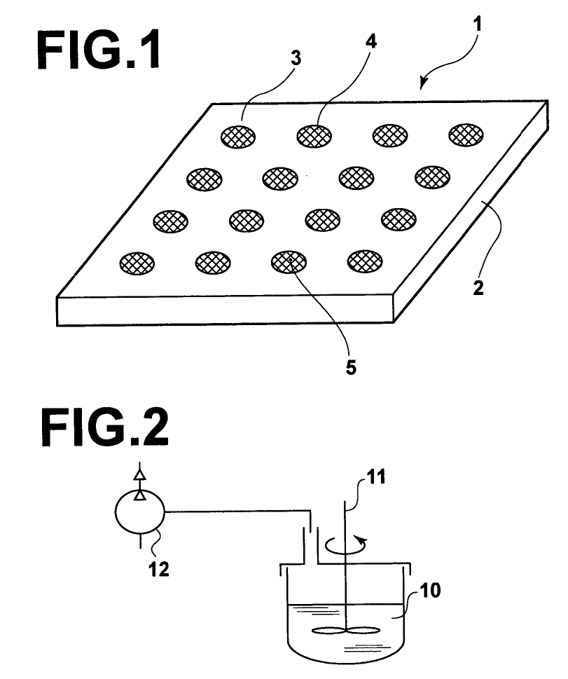

FIG. 1 is a schematic perspective view showing an example of a biochemical analysis unit utilized for the assay method using a biochemical analysis unit in accordance with the present invention. With reference to FIG. 1, a biochemical analysis unit 1 comprises a base plate 2, which is provided with a plurality of holes 3, 3, . . . , and a plurality of adsorptive regions 4, 4, . . . , each of which is filled in one of the holes 3, 3, . . . and comprises a porous material adhered to the base plate 2. Each of ligands or receptors, whose structures or characteristics are known, has been spotted onto one of the adsorptive regions 4, 4, . . . and has then been immobilized with treatment.

Such that scattering may be prevented from occurring within the biochemical analysis unit 1, the base plate 2 should preferably be made from a material, which does not transmit radiation or light or which attenuates the radiation or the light. The material for the formation of the base plate 2 should preferably be a metal or a ceramic material. Also, in cases where a plastic material, for which the hole making processing is capable of being performed easily, is employed as the material for the formation of the base plate 2, particles should preferably be dispersed within the plastic material, such that the radiation or the light is capable of being attenuated even further.

Examples of the metals, which may be utilized preferably for the formation of the base plate 2, include copper, silver, gold, zinc, lead, aluminum, titanium, tin, chromium, iron, nickel, cobalt, tantalum, and alloys, such as stainless steel and bronze. Examples of the ceramic materials, which may be utilized preferably for the formation of the base plate 2, include alumina, zirconia, magnesia, and quartz. Examples of the plastic materials, which may be utilized preferably for the formation of the base plate 2, include polyolefins, such as a polyethylene and a polypropylene; polystyrenes; acrylic resins, such as a polymethyl methacrylate; polyvinyl chlorides; polyvinylidene chlorides; polyvinylidene fluorides; polytetrafluoroethylenes; polychlorotrifluoroethylenes; polycarbonates; polyesters, such as a polyethylene naphthalate and a polyethylene terephthalate; aliphatic polyamides, such as a 6-nylon and a 6,6-nylon; polyimides; polysulfones; polyphenylene sulfides; silicon resins, such as a polydiphenyl siloxane; phenolic resins, such as novolak; epoxy resins; polyurethanes; celluloses, such as cellulose acetate and nitrocellulose; copolymers, such as a butadiene-styrene copolymer; and blends of plastic materials.

Such that the density of the holes 3, 3, . . . made through the base plate 2 may be enhanced, the area (size) of the opening of each of the holes 3, 3, . . . may ordinarily be smaller than 5 mm². The area of the opening of each of the holes 3, 3, . . . should preferably be smaller than 1 mm², should more preferably be smaller than 0.3 mm², and should most preferably be smaller than 0.01 mm². Also, the area of the opening of each of the holes 3, 3, . . . should preferably be at least 0.001 mm².

The pitch of the holes 3, 3, . . . (i.e., the distance between the center points of two holes which are adjacent to each other) should preferably fall within the range of 0.05 mm to 3 mm. Also, the spacing between two adjacent holes 3, 3 (i.e., the shortest distance between edges of two adjacent holes 3, 3) should preferably fall within the range of 0.01 mm to 1.5 mm. The number (the array density) of the holes 3, 3, . . . may ordinarily be at least 10 holes/cm². The number (the array density) of the holes 3, 3, . . . should preferably be at least 100 holes/cm², should more preferably be at least 500 holes/cm², and should most preferably be at least 1,000 holes/cm². Also, the number (the array density) of the holes 3, 3, . . . should preferably be at most 100,000 holes/cm², and should more preferably be at most 10,000 holes/cm². The holes 3, 3, . . . need not necessarily be arrayed at equal spacing as illustrated in FIG. 1. For example, the holes 3, 3, . . . may be grouped into several number of blocks (units) comprising a plurality of holes and may be formed in units of the blocks.

In the assay method using a biochemical analysis unit in accordance with the present invention, as the porous material for the formation of the adsorptive regions of the biochemical analysis unit, a porous quality material or a fiber material may be utilized preferably. The porous quality material and the fiber material may be utilized in combination in order to form the adsorptive regions of the biochemical analysis unit. In the assay method using a biochemical analysis unit in accordance with the present invention, the porous material, which may be utilized for the formation of the adsorptive regions of the biochemical analysis unit, may be an organic material, an inorganic material, or an organic-inorganic composite material.

The organic porous quality material, which may be utilized for the formation of the adsorptive regions of the biochemical analysis unit, may be selected from a wide variety of materials. However, the organic porous quality material should preferably be a carbon porous quality material, such as active carbon, or a porous quality material capable of forming a membrane filter. As the porous quality material capable of forming a membrane filter, a polymer soluble in a solvent should preferably be utilized. Examples of the polymers soluble in a solvent include cellulose derivatives, such as nitrocellulose, regenerated cellulose, cellulose acetate, and cellulose acetate butyrate; aliphatic polyamides, such as a 6-nylon, a 6,6-nylon, and a 4,10-nylon; polyolefins, such as a polyethylene and a polypropylene; chlorine-containing polymers, such as a polyvinyl chloride and a polyvinylidene chloride; fluorine resins, such as a polyvinylidene fluoride and a polytetrafluoride; polycarbonates; polysulfones; alginic acids and alginic acid derivatives, such as alginic acid, calcium alginate, and an alginic acid-polylysine polyion complex; and collagen. Copolymers or composite materials (mixture materials) of the above-enumerated polymers may also be utilized.

The fiber material, which may be utilized for the formation of the adsorptive regions of the biochemical analysis unit, maybe selected from a wide variety of materials. Examples of the fiber materials, which may be utilized preferably, include the cellulose derivatives and the aliphatic polyamides enumerated above.

The inorganic porous quality material, which may be utilized for the formation of the adsorptive regions of the biochemical analysis unit, may be selected from a wide variety of materials. Examples of the inorganic porous quality materials, which may be utilized preferably, include metals, such as platinum, gold, iron, silver, nickel, and aluminum; oxides of metals, and the like, such as alumina, silica, titania, and zeolite; metal salts, such as hydroxyapatite and calcium sulfate; and composite materials of the above-enumerated materials.

Perforation of the plurality of the holes 3, 3, . . . through the base plate 2 may be performed with, for example, a punching technique for punching with a pin, a technique for electrical discharge machining, in which a pulsed high voltage is applied across electrodes in order to volatilize the base plate material, an etching technique, or a laser beam irradiation technique. In cases where the material of the base plate is a metal material or a plastic material, the biochemical analysis unit may be prepared with an operation for performing corona discharge or plasma discharge on the surface of the base plate, applying an adhesive agent to the surface of the base plate, and laminating the porous material for the formation of the adsorptive regions by use of means, such as a press. At the time of the lamination, the porous material for the formation of the adsorptive regions may be heated and softened, such that the adsorptive regions may be formed easily within the holes. Also, in cases where the porous material for the formation of the adsorptive regions is pressed against the base plate, the base plate and the porous material for the formation of the adsorptive regions may be divided previously into a plurality of sheets, and the plurality of the sheets may be pressed intermittently. Alternatively, a long web of the base plate and a long web of the porous material for the formation of the adsorptive regions may be conveyed continuously between two rolls.

In the assay method using a biochemical analysis unit in accordance with the present invention, the biochemical analysis unit having been prepared by use of the material and the technique described above may be utilized. Alternatively, a commercially available biochemical analysis unit may be utilized. It is also possible to utilize a biochemical analysis unit, in which the ligands or the receptors have already been bound respectively to the porous adsorptive regions.

The assay method using a biochemical analysis unit in accordance with the present invention comprises the steps of:

i) obtaining a biochemical analysis unit provided with a plurality of porous adsorptive regions, to which ligands or receptors have been bound respectively, and

ii) performing a specific binding detecting process comprising the steps of:

a) forcibly causing a receptor or a ligand to flow such that the receptor or the ligand flows across each of the porous adsorptive regions of the biochemical analysis unit, the receptor or the ligand being thus subjected to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and

b) detecting the receptor or the ligand, which has thus been specifically bound to at least one of the ligands or at least one of the receptors, by the utilization of a labeling substance,

a liquid being forcibly caused to flow, such that the liquid flows across each of the porous adsorptive regions of the biochemical analysis unit, during the specific binding detecting process,

wherein a liquid, which has been subjected to gas content decreasing processing for decreasing the content of a dissolved gas, is employed as the liquid, which is forcibly caused to flow.

As a first example, the specific binding detecting process may comprise the steps of:

a) forcibly causing a reaction liquid containing a labeled receptor or a labeled ligand, which has been labeled with a labeling substance, to flow such that the reaction liquid flows across each of the porous adsorptive regions of the biochemical analysis unit provided with the plurality of the porous adsorptive regions, to which the ligands or the receptors have been bound respectively, the labeled receptor or the labeled ligand being thus subjected to the specific binding with the ligands or the receptors, each of which has been bound to one of the porous adsorptive regions of the biochemical analysis unit, the labeled receptor or the labeled ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and

b) detecting the labeled receptor or the labeled ligand, which has thus been specifically bound to at least one of the ligands or at least one of the receptors, by the utilization of the labeling substance.

Examples of the ligands or the receptors, which are bound respectively to the porous adsorptive regions of the biochemical analysis unit, include hormones, tumor markers, enzymes, antibodies, antigens, abzymes, other proteins, nucleic acids, cDNA's, DNA's, and RNA's, whose characteristics, compositions, structures, base sequences, base lengths, and the like, are known.

The labeled receptor or the labeled ligand is the substance, which has been sampled from an organism through extraction, isolation, or the like, or has been subjected to chemical treatment after being sampled, and which has been labeled with the labeling substance. The labeled receptor or the labeled ligand is capable of undergoing the specific binding with at least one of the ligands, each of which has been bound to one of the porous adsorptive regions of the biochemical analysis unit, or at least one of the receptors, each of which has been bound to one of the porous adsorptive regions of the biochemical analysis unit. Examples of the labeled receptors or the labeled ligands include hormones, tumor markers, enzymes, antibodies, antigens, abzymes, other proteins, nucleic acids, DNA's, and mRNA's.

The labeling substance may be a substance, which is capable of producing radiation by itself, a substance, which is capable of emitting light by itself, a substance, which is capable of forming a color by itself, or a substance, which is capable of producing fluorescence by itself when being exposed to light. Alternatively, the labeling substance may be a substance, which is capable of causing a chemical substance to emit light, to form a color, or to produce the fluorescence through, for example, decomposition or reaction of the chemical substance when being brought into contact with the chemical substance. As for the former type of the labeling substance, a radioactive isotope (RI) maybe employed as the radiation producing labeling substance. Also, an acridinium ester, or the like, may be employed as the light emitting labeling substance. Further, gold colloidal particles, or the like, may be employed as the color forming labeling substance. Furthermore, fluorescein, or the like, maybe employed as the fluorescent labeling substance. As the latter type of the labeling substance, an enzyme may be employed. Examples of the enzymes include alkaline phosphatase, peroxidase, luciferase, and p-galactosidase. When one of the above-enumerated enzymes acting as the labeling substance is brought into contact with a chemical luminescence substrate, a dye substrate, or a fluorescence substrate, the enzyme is capable of causing the chemical luminescence substrate to produce the chemical luminescence, causing the dye substrate to form a color, or causing the fluorescence substrate to produce the fluorescence.

By way of example, in cases where the enzyme is alkaline phosphatase, peroxidase, or luciferase, the chemical luminescence substrate maybe dioxetane, luminol, or luciferin, respectively. In cases where the enzyme is alkaline phosphatase, the dye substrate may be p-nitrophenyl phosphate. In cases where the enzyme is β-galactosidase, the dye substrate may be p-nitrophenyl-β-D-galactoside, or the like. In cases where the enzyme is alkaline phosphatase, the fluorescence substrate may be 4-methylumbellifer phosphoric acid. In cases where the enzyme is peroxidase, the fluorescence substrate may be 3-(4-hydroxyphenyl)-propionic acid. In cases where the enzyme is β-galactosidase, the fluorescence substrate may be 4-methylumbellifer-β-D-galactoside, or the like.

As a second example, the specific binding detecting process may comprise the steps of:

a) subjecting the receptor or the ligand to the specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,

b) forcibly causing a reaction liquid containing a labeled body, which has been labeled with a labeling substance, to flow such that the reaction liquid flows across each of the porous adsorptive regions of the biochemical analysis unit, the labeled body being thus specifically bound to the receptor or the ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and

c) detecting the receptor or the ligand, which has been specifically bound to at least one of the ligands or at least one of the receptors, by the utilization of the labeled body.

The aforesaid second example of the specific binding detecting process is the so-called sandwich technique, wherein the receptor or the ligand, which is to be detected, is sandwiched between the ligand or the receptor, which has been bound to the adsorptive region, and the labeled body. In this case, the receptor or the ligand, which is to be detected, is the substance, which has been sampled from an organism through extraction, isolation, or the like, or has been subjected to chemical treatment after being sampled, and which has been labeled with the labeling substance. The receptor or the ligand is capable of undergoing the specific binding with at least one of the ligands, each of which has been bound to one of the porous adsorptive regions of the biochemical analysis unit, or at least one of the receptors, each of which has been bound to one of the porous adsorptive regions of the biochemical analysis unit. Examples of the receptors or the ligands, which are to be detected, include hormones, tumor markers, enzymes, antibodies, antigens, abzymes, other proteins, nucleic acids, DNA's, and mRNA's.

The labeled body, which has been labeled with the labeling substance, is a body, which has been labeled with the labeling substance described above and is capable of undergoing the specific binding with a reaction site of the receptor or the ligand, which is to be detected. Examples of the labeled bodies include antigens, antibodies, hormones, tumor markers, enzymes, abzymes, other proteins, nucleic acids, cDNA's, DNA's, and RNA's, whose characteristics, compositions, structures, base sequences, base lengths, and the like, are known.

As a third example, the specific binding detecting process may comprise the steps of:

a) subjecting an auxiliary substance-bound receptor or an auxiliary substance-bound ligand, to which an auxiliary substance has been bound, to the specific binding with the ligands or the receptors, each of which has been bound to one of the porous adsorptive regions of the biochemical analysis unit, the auxiliary substance-bound receptor or the auxiliary substance-bound ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,

b) forcibly causing a reaction liquid containing a labeling substance, which is capable of undergoing specific binding with the auxiliary substance, to flow such that the reaction liquid flows across each of the porous adsorptive regions of the biochemical analysis unit, the labeling substance, which is capable of undergoing specific binding with the auxiliary substance, being thus specifically bound to the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and

c) detecting the auxiliary substance-bound receptor or the auxiliary substance-bound ligand, which has been specifically bound to at least one of the ligands or at least one of the receptors, by the utilization of the labeling substance.

The auxiliary substance is a substance capable of undergoing the binding with the labeling substance. Examples of preferable auxiliary substances include antigens, such as digoxigenin, biotin, avidin, and fluorescein, and antibodies with respect to the above-enumerated antigens. Also, the auxiliary substance may be a biological binding partner, such as avidin with respect to biotin. In this case, the labeling substance capable of binding is a substance, which is capable of undergoing the specific binding with the auxiliary substance and has been labeled with the labeling substance described above.

As an embodiment of the assay method using a biochemical analysis unit in accordance with the present invention, an assay method with a chemical luminescence method will be described hereinbelow. With the chemical luminescence method using a biochemical analysis unit in accordance with the present invention, the ligands or the receptors are fixed respectively to the adsorptive regions of the biochemical analysis unit. Also, an antigen-bound receptor or an antigen-bound ligand, which has been labeled with an antigen (acting as the auxiliary substance), is subjected to the hybridization, or the like, with the ligands or the receptors, each of which has been fixed to one of the adsorptive regions of the biochemical analysis unit, and the antigen-bound receptor or the antigen-bound ligand is thus specifically bound to at least one of the ligands or at least one of the receptors. Thereafter, an antibody with respect to the antigen, with which the antigen-bound receptor or the antigen-bound ligand has been labeled, is labeled with an enzyme, which is capable of causing a chemical luminescence substrate to produce the chemical luminescence. (The antibody having been labeled with an enzyme, which is capable of causing the chemical luminescence substrate to produce the chemical luminescence, will hereinbelow referred to as the enzyme-labeled antibody.) The enzyme-labeled antibody is subjected to the specific binding with the antigen of the antigen-bound receptor or the antigen-bound ligand. Further, the enzyme-labeled antibody, which has been specifically bound to the antigen of the antigen-bound receptor or the antigen-bound ligand, is brought into contact with the chemical luminescence substrate, which is capable of undergoing the specific binding with the enzyme of the enzyme-labeled antibody. The chemical luminescence having wavelengths falling within the visible light wavelength range, which chemical luminescence is produced by the chemical luminescence substrate when the chemical luminescence substrate is brought into contact with the enzyme of the enzyme-labeled antibody, is detected photoelectrically.

Specifically, with the chemical luminescence method using a biochemical analysis unit in accordance with the present invention, firstly, the ligands or the receptors are bound respectively to the adsorptive regions of the biochemical analysis unit, which is provided with the plurality of the porous adsorptive regions.

The ligands or the receptors, which are bound respectively to the porous adsorptive regions of the biochemical analysis unit, may be of the kinds described above. After the ligands or the receptors have been spotted respectively onto the adsorptive regions of the biochemical analysis unit, the ligands or the receptors are capable of being fixed to the adsorptive regions with ultraviolet light irradiation, or the like. In cases where the aforesaid biochemical analysis unit, in which the ligands or the receptors have already been bound respectively to the porous adsorptive regions, is utilized, the steps of spotting and fixing the ligands or the receptors are omitted.

Thereafter, the antigen-bound receptor or the antigen-bound ligand is subjected to the specific binding with the ligands or the receptors, each of which has been bound to one of the porous adsorptive regions of the biochemical analysis unit. The antigen-bound receptor or the antigen-bound ligand is thus specifically bound to at least one of the ligands or at least one of the receptors. Before the antigen-bound receptor or the antigen-bound ligand is thus subjected to the specific binding with the ligands or the receptors, a reaction liquid containing the antigen-bound receptor or the antigen-bound ligand is subjected to the gas content decreasing processing for decreasing the content of a gas dissolved in the reaction liquid. As an example of an apparatus for performing the gas content decreasing processing for decreasing the content of the dissolved gas, a deaerator illustrated in FIG. 2 or a deaerator illustrated in FIG. 3 may be employed.

FIG. 2 is a schematic view showing an example of a batch type of deaerator. With the deaerator shown in FIG. 2, a reaction liquid 10 having been introduced into a predetermined vessel is stirred gently with a stirrer 11 such that uniform vapor-liquid interface maybe kept, and the pressure within the vessel is set at a negative pressure lower than the atmospheric pressure by use of a vacuum pump 12. In this manner, the reaction liquid 10 is deaerated.

FIG. 3 is a schematic view showing an example of a continuous type of deaerator. With the deaerator shown in FIG. 3, the pressure of the exterior of a tube 14 (made from, for example, Teflon (trade name)) is set at a negative pressure lower than the atmospheric pressure by use of a vacuum pump 15. Also, the reaction liquid 10 is fed into the tube 14 by use of a pump 13, and a gas dissolved in the reaction liquid 10 is allowed to be removed from the reaction liquid 10 through the wall of the tube 14.

In both the cases of the batch type of the deaerator and the continuous type of the deaerator, during the deaeration, the negative pressure should preferably be set at a temperature higher than the temperature, at which the reaction liquid is to be subjected to the reaction. The content of the dissolved gas should preferably be decreased to approximately one tenth of the saturated solubility. Besides the reaction liquid containing the antigen-bound receptor or the antigen-bound ligand, all of the liquids, each of which is to be forcibly caused to flow such that the liquid flows across each of the adsorptive regions, may be subjected to the gas content decreasing processing for decreasing the content of the dissolved gas.

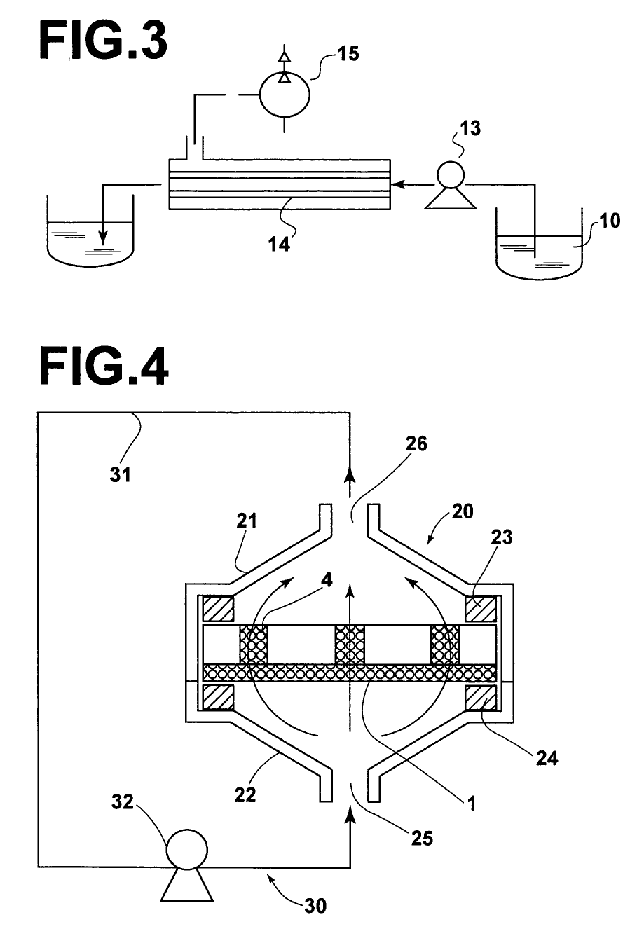

After the gas content decreasing processing for decreasing the content of the dissolved gas in the reaction liquid containing the antigen-bound receptor or the antigen-bound ligand has been performed, the biochemical analysis unit is set within, for example, a reaction vessel, which is illustrated in FIG. 4 and in which the reaction liquid is capable of being forcibly caused to flow such that the reaction liquid flows across each of the adsorptive regions of the biochemical analysis unit. In this state, the antigen-bound receptor or the antigen-bound ligand is subjected to the specific binding with the ligands or the receptors, each of which has been bound to one of the porous adsorptive regions of the biochemical analysis unit.

FIG. 4 is a schematic view showing an example of a biochemical analysis apparatus (a reactor) utilized for the assay method using a biochemical analysis unit in accordance with the present invention. With reference to FIG. 4, the reactor comprises a reaction vessel 20 and flowing means 30. The reaction vessel 20 comprises a reaction vessel upper half 21 and a reaction vessel lower half 22. The reaction vessel upper half 21 is releasably secured to the reaction vessel lower half 22. Also, the reaction vessel 20 is provided with a support section for releasably supporting the biochemical analysis unit 1 within the reaction vessel 20, the biochemical analysis unit 1 being provided with the plurality of the porous adsorptive regions, to which the ligands or the receptors have been bound respectively. The support section comprises an upper support piece 23 and a lower support piece 24. When the biochemical analysis unit 1 is to be set within the reaction vessel 20, the reaction vessel upper half 21 is dismounted from the reaction vessel lower half 22, and the biochemical analysis unit 1 is set on the lower support piece 24. A bottom wall of the reaction vessel lower half 22 is provided with a reaction liquid inlet 25, through which a reaction liquid is capable of flowing. Also, a top wall of the reaction vessel upper half 21 is provided with a reaction liquid outlet 26, through which the reaction liquid is capable of flowing.

The flowing means 30 comprises a reaction liquid circulating pipe 31 and a pump 32. One end of the reaction liquid circulating pipe 31 is releasably fitted to the reaction liquid inlet 25 of the reaction vessel 20. The other end of the reaction liquid circulating pipe 31 is releasably fitted to the reaction liquid outlet 26 of the reaction vessel 20. The reaction liquid is introduced by the pump 32 into the reaction vessel 20 through the reaction liquid inlet 25. Within the reaction vessel 20, the reaction liquid is forcibly caused to flow such that the reaction liquid flows across each of the adsorptive regions 4, 4, . . . of the biochemical analysis unit 1. Thereafter, the reaction liquid is discharged through the reaction liquid outlet 26, passes through the reaction liquid circulating pipe 31, and circulates through the reaction vessel 20.

With the gas content decreasing processing for decreasing the content of the dissolved gas in the reaction liquid, the occurrence of bubbles in the reaction liquid due to cavitation is capable of being suppressed. However, there is the risk that a gas, which is present in fine voids of the adsorptive regions of the biochemical analysis unit, will form bubbles. Also, there is the risk that, at the time of change-over between the reaction liquid and a washing liquid, bubbles will mix into the liquid. Therefore, the reactor should preferably be provided with bubble removing means for removing bubbles or bubble dissolving means for dissolving bubbles.

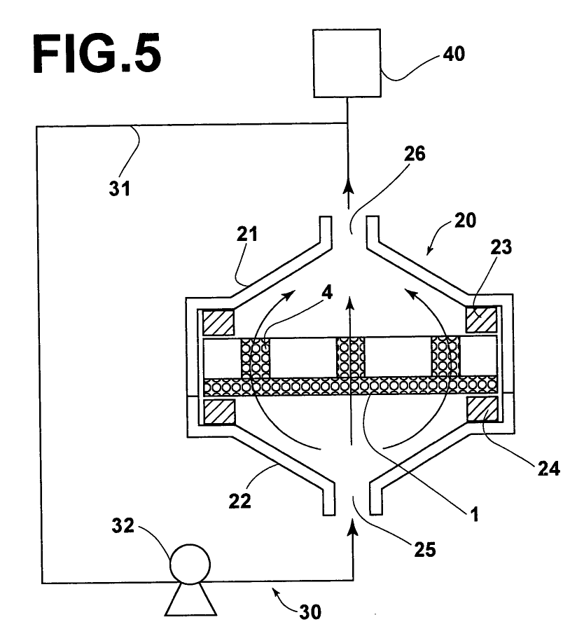

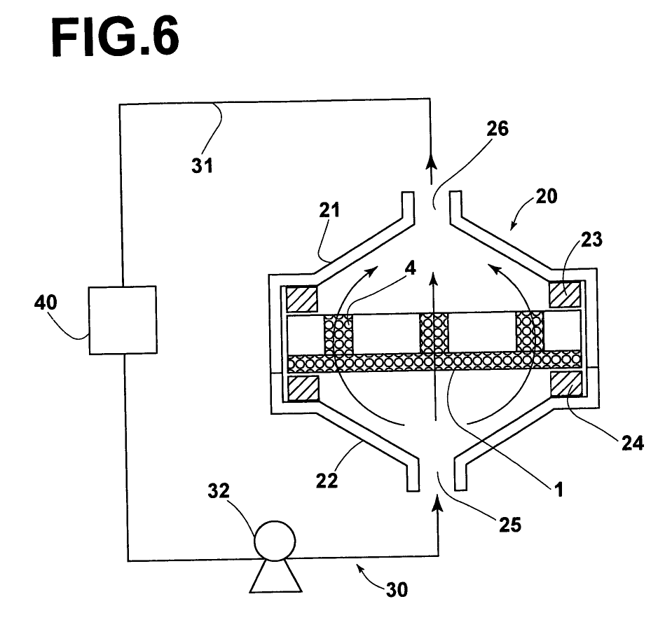

FIG. 5 is a schematic view showing an embodiment of the biochemical analysis apparatus in accordance with the present invention. The biochemical analysis apparatus illustrated in FIG. 5 comprises bubble removing means 40, which is located at a branch of the reaction liquid circulating pipe 31. The reaction liquid, which has passed through the reaction liquid outlet 26, is introduced into the bubble removing means 40, which is located at the branch of the reaction liquid circulating pipe 31, and the bubble removing means 40 removes the bubbles, which have occurred due to the flowing, from the reaction liquid. The bubble removing means 40 may be constituted of, for example, a net or a filter, which is capable of catching the bubbles. In the example of FIG. 5, the bubble removing means 40 is located at the branch of the reaction liquid circulating pipe 31. Alternatively, as illustrated in FIG. 6, the bubble removing means 40 maybe located at an intermediate point of the piping of the reaction liquid circulating pipe 31. Also, bubble dissolving means may be utilized in lieu of the bubble removing means 40 or in addition to the bubble removing means 40. As in the cases of the bubble removing means 40, the bubble dissolving means may be located at the branch of the reaction liquid circulating pipe 31 or at an intermediate point of the piping of the reaction liquid circulating pipe 31. The bubble dissolving means may be constituted of, for example, an ultrasonic wave irradiating tank.

Each of the biochemical analysis apparatuses illustrated in FIG. 4, FIG. 5, and FIG. 6 is the reactor constituted such that the reaction liquid is circulated through the biochemical analysis unit 1. Alternatively, a biochemical analysis apparatus may be utilized, in which the reaction liquid is not circulated. For example, a biochemical analysis apparatus may be utilized, in which the reaction liquid is caused to undergo reciprocal flowing across the biochemical analysis unit 1. Also, a biochemical analysis apparatus maybe utilized, in which the reaction liquid merely passes through the biochemical analysis unit 1 from below (or from above).

In order for the antigen-bound receptor or the antigen-bound ligand, which has not been specifically bound to the ligands or the receptors having been bound respectively to the porous adsorptive regions of the biochemical analysis unit, to be removed, the biochemical analysis unit having been set within the reaction vessel should preferably be washed with a technique for forcibly causing a washing liquid to flow across each of the adsorptive regions. In such cases, since the washing liquid is forcibly caused to flow across each of the adsorptive regions, the antigen-bound receptor or the antigen-bound ligand, which has not been specifically bound to the ligands or the receptors having been bound respectively to the porous adsorptive regions of the biochemical analysis unit, is capable of being peeled off and removed efficiently. Therefore, the washing efficiency is capable of being enhanced markedly.

As will be described later, the reaction liquid, which contains the enzyme-labeled antibody, is forcibly caused to flow such that the reaction liquid flows across each of the adsorptive regions of the biochemical analysis unit, and the enzyme-labeled antibody is thus subjected to the specific binding with the antigen-bound receptor or the antigen-bound ligand. After the enzyme-labeled antibody has thus been subjected to the specific binding with the antigen-bound receptor or the antigen-bound ligand, the enzyme-labeled antibody, which has not been specifically bound to the antigen-bound receptor or the antigen-bound ligand, may be removed. In cases where the enzyme-labeled antibody, which has not been specifically bound to the antigen-bound receptor or the antigen-bound ligand, is to be removed, the washing process described above should preferably be performed. In this manner, the enzyme-labeled antibody, which has not been specifically bound to the antigen-bound receptor or the antigen-bound ligand, is capable of being peeled off and removed efficiently. Therefore, the washing efficiency is capable of being enhanced markedly.

After the antigen-bound receptor or the antigen-bound ligand has been specifically bound to at least one of the ligands or the receptors, each of which has been bound to one of the porous adsorptive regions of the biochemical analysis unit, in the manner described above, the reaction liquid, which contains the enzyme-labeled antibody, is forcibly caused to flow such that the reaction liquid flows across each of the adsorptive regions of the biochemical analysis unit, and the enzyme-labeled antibody is thus subjected to the specific binding with the antigen-bound receptor or the antigen-bound ligand. Before the enzyme-labeled antibody is thus subjected to the specific binding with the antigen-bound receptor or the antigen-bound ligand, the adsorptive regions should preferably be blocked with a blocking process, wherein a blocking buffer with respect to the enzyme-labeled antibody is forcibly caused to flow such that the blocking buffer flows across each of the adsorptive regions. Also, the specific binding of the enzyme-labeled antibody with the antigen-bound receptor or the antigen-bound ligand should preferably be performed with a process, wherein the enzyme-labeled antibody is added to the blocking buffer with respect to the enzyme-labeled antibody, and the blocking buffer containing the enzyme-labeled antibody is forcibly caused to flow such that the blocking buffer flows across each of the adsorptive regions. In such cases, the intensity of the background of the adsorptive regions is capable of being suppressed even further.

Thereafter, the biochemical analysis unit is taken out from the reaction vessel, and a chemical luminescence substrate is brought into contact with the enzyme-labeled antibody, which has been specifically bound to the antigen-bound receptor or the antigen-bound ligand. In cases where the chemical luminescence substrate and the enzyme are thus brought into contact with each other, the chemical luminescence having wavelengths falling within the visible light wavelength range is produced from the corresponding adsorptive region. Therefore, the produced chemical luminescence may be detected photoelectrically, and the image data for a biochemical analysis may be formed in accordance with the detected chemical luminescence. In this manner, the antigen-bound receptor or the antigen-bound ligand is capable of being detected and determined.

The present invention will further be illustrated by the following nonlimitative example.

### EXAMPLE 1

Through-holes, each of which had a size of 0.0007 cm², were formed in base plate constituted of a SUS304 sheet (acting as a base plate material sheet) having a thickness of 100 μm. The through-holes were formed at a density of 1,600 holes per 18 mm×18 mm.

Thereafter, an adhesive agent was applied to one surface of the base plate material sheet, and the adhesive agent, which entered into the holes having been formed in the base plate material sheet, was removed by suction. The adhesive agent remaining on the surface of the base plate material sheet was then dried. Thereafter, a 6,6-nylon membrane having a pore size of 0.45 μm and a thickness of 170 μm was superposed upon the surface of the base plate material sheet, which surface had been coated with the adhesive agent. The combination of the 6,6-nylon membrane and the base plate material sheet was then heated to a temperature of 150° C. and pressed under pressure such that the pressure per 1 cm² was 300 kg. The 6,6-nylon membrane was thus press-fitted into the fine holes of the base plate material sheet. In this manner, a biochemical analysis unit, which comprised a stainless steel barrier wall and the plurality of polymer-filled regions formed in the fine holes, was prepared.

Also, a pBR328-DNA liquid having a concentration of 1 ng/μl (supplied by Roche Diagnostics K.K.) was subjected to thermal denaturation, and the pBR328-DNA was thus converted into a single stranded form. Thereafter, 10 nl of the pBR328-DNA liquid was spotted onto each of the adsorptive regions of the biochemical analysis unit having been prepared in the manner described above. Thereafter, with irradiation of ultraviolet light (254 nm, 33 mJ/cm²), the single stranded pBR328/BgII, HinfI was fixed to the adsorptive regions of the biochemical analysis unit.

Thereafter, a hybridization buffer, which contained 50 ml of a 1M phosphoric acid buffer solution (a solution containing 7.1 g of anhydrous disodium phosphate per 100 ml and having a pH value adjusted at 7.2 by the addition of phosphoric acid), 43 ml of sterilized deionized water, and 7 g of a dodecyl sulfonic acid sodium salt per 100 ml, was prepared. The thus prepared hybridization buffer was introduced into the deaerator shown in FIG. 2 and subjected to a deaeration process, wherein the pressure within the deaerator was set at a negative pressure of 79.8 kPa (600 Torr) with respect to the atmospheric pressure, the temperature was raised to 68° C., and the deaeration was performed for 10 minutes. The concentration of the dissolved oxygen in the hybridization buffer after being subjected to the deaeration process was 1.5 (mg/l).

Also, a digoxigenin-labeled pBR328-DNA liquid (5 ng/μl) was diluted with a TE buffer, and the concentration of the digoxigenin-labeled pBR328-DNA liquid was thus set at a predetermined value. Thermal denaturation was then performed, and the digoxigenin-labeled pBR328-DNA was thus converted into a single stranded form. Thereafter, the digoxigenin-labeled pBR328-DNA liquid was diluted even further by the addition of the deaerated hybridization buffer. In this manner, the digoxigenin-labeled pBR328-DNA liquid having a predetermined concentration (a hybridization reaction liquid) was prepared.

Thereafter, the biochemical analysis unit was set in the reaction vessel of the biochemical analysis apparatus illustrated in FIG. 5. Also, the hybridization buffer was fed into the reaction vessel, in which the biochemical analysis unit had been accommodated. The pump of the biochemical analysis apparatus was actuated, and a hybridization reaction was performed for 18 hours, while the temperature of the reaction vessel and the temperature of the hybridization reaction liquid were being kept at 68° C. After the hybridization reaction was finished, a washing liquid was fed into the reaction vessel, the pump was actuated, and the adsorptive regions of the biochemical analysis unit were thus washed.

A washing buffer (supplied by Roche Diagnostics K.K.), which had been diluted with sterilized deionized water to a concentration of 1/10, was employed as a washing liquid, and the washing liquid was fed into the reaction vessel, in which the biochemical analysis unit had been accommodated. The pump was actuated, and the liquid in the adsorptive regions of the biochemical analysis unit was replaced by the washing liquid.

Thereafter, by use of a maleic acid buffer (supplied by Roche Diagnostics K.K.), which had been diluted with sterilized deionized water to a concentration of 1/10, a blocking buffer solution ((supplied by Roche Diagnostics K.K.) was diluted to a concentration of 1/10. The thus diluted blocking buffer solution was then subjected to filtration with a polyether sulfone filter (pore diameter: 0.2 μm) and then utilized as a blocking agent. The blocking agent was fed into the reaction vessel, and the pump was driven for 10 minutes. In this manner, the blocking agent was caused to permeate through all parts of the adsorptive regions of the biochemical analysis unit. Thereafter, the operation of the pump was ceased, and the blocking agent was allowed to stand for 50 minutes within the reaction vessel.

Thereafter, an anti-digoxigenin-AP-conjugate (an alkaline phosphatase-labeled digoxigenin antibody) was subjected to centrifugal filtration with a polyvinylidene fluoride filter (pore diameter: 0.22 μm). The anti-digoxigenin-AP-conjugate having been collected by filtration was then diluted with the aforesaid blocking agent to a concentration of 1/10,000, and an enzyme-labeled antibody liquid was thereby prepared. The thus prepared enzyme-labeled antibody liquid was fed into the reaction vessel, and the pump was driven for one minute. In this manner, the enzyme-labeled antibody liquid was caused to permeate through all parts of the adsorptive regions of the biochemical analysis unit, and an antigen-antibody reaction was performed. Thereafter, the operation of the pump was ceased, and the enzyme-labeled antibody liquid was allowed to stand for one hour within the reaction vessel.

After the antigen-antibody reaction was completed, the washing liquid was fed into the reaction vessel. Also, the pump was driven, the washing buffer was thus caused to permeate through all parts of the adsorptive regions of the biochemical analysis unit, and the adsorptive regions of the biochemical analysis unit were thus washed. The biochemical analysis unit was taken out from the reaction vessel and was then brought into contact with a liquid containing a chemical luminescence substrate (CDP-star, ready to use, supplied by Roche Diagnostics K.K.). Also, the chemical luminescence, which was emitted from the adsorptive regions of the biochemical analysis unit, was detected photoelectrically by use of a cooled CCD camera (LAS1000, supplied by Fuji Photo Film Co., Ltd.). In this manner, a digital signal was formed.

### COMPARATIVE EXAMPLE 1

The chemical luminescence operations were performed in the same manner as that in Example 1, except that the deaeration of the hybridization buffer was not performed (the concentration of the dissolved oxygen in the hybridization buffer: 8 (mg/l)), and the biochemical analysis apparatus illustrated in FIG. 4 was utilized. In this manner, the intensity of the digital signal and the intensity of the background were detected.

With respect to the amounts of the digoxigenin-labeled pBR328 contained in the hybridization buffer reaction liquid, the detection results listed in Table 1 below were obtained.

TABLE 1

| | Example 1 | | | |
|---|---|---|---|---|
| Amount | Mean | | Comparative Example 1 | |
| (pg) of | value of | Variation (%) | Mean value | Variation (%) |
| digoxigenin- | signal- | in | of | in |
| labeled | to-noise | signal-to-noise | signal-to-noise | signal-to-noise |
| pBR328 | ratio | ratio | ratio | ratio |
| 0.1 | 2.44 | 22.4 | 1.1 | 45.2 |
| 0.5 | 15.1 | 14.2 | 3.6 | 27.4 |
| 5 | 99.4 | 11.7 | 35.2 | 21.3 |

As clear from Table 1, in Example 1, in which the deaeration of the hybridization buffer was performed, and in which the reaction was performed by use of the biochemical analysis apparatus provided with the bubble removing means, the signal-to-noise ratio was higher in every case, regardless of the concentration of the digoxigenin-labeled pBR328, than in Comparative Example 1, in which the deaeration of the hybridization buffer was not performed, and in which the reaction was performed by use of the biochemical analysis apparatus that was not provided with the bubble removing means. Also, in Example 1, the variation in signal-to-noise ratio for different positions of the adsorptive regions was capable of being kept smaller than in Comparative Example.

As described above, with the assay method using a biochemical analysis unit in accordance with the present invention, wherein the liquid is forcibly caused to flow such that the liquid flows across each of the adsorptive regions of the biochemical analysis unit, the liquid, which has been subjected to the gas content decreasing processing for decreasing the content of the dissolved gas, is employed as the liquid, which is forcibly caused to flow. Alternatively, with the assay method using a biochemical analysis unit in accordance with the present invention, the bubble removing processing for removing bubbles, which are present in the liquid, from the liquid is performed during the flowing of the liquid. As another alternative, with the assay method using a biochemical analysis unit in accordance with the present invention, the bubble dissolving processing for dissolving bubbles, which are present in the liquid, is performed during the flowing of the liquid. Therefore, with the assay method using a biochemical analysis unit in accordance with the present invention, the occurrence of bubbles due to cavitation is capable of being suppressed. Accordingly, the problems are capable of being prevented from occurring in that the bubbles cling to the surfaces of the adsorptive regions and cause the flow of the liquid to be biased. As a result, the problems are capable of being prevented from occurring in that the signal-to-noise ratio becomes low, and in that the signal-to-noise varies for different positions of the adsorptive regions. Also, the problems are capable of being prevented from occurring in that the bubbles clinging to the surfaces of the adsorptive regions obstruct the detection of the receptor or the ligand.

What is claimed is:

1. An assay method using a biochemical analysis unit, comprising the steps of:
i) obtaining a biochemical analysis unit provided with a plurality of porous adsorptive regions, to which ligands or receptors have been bound respectively, and
ii) performing a specific binding detecting process comprising the steps of:
a) forcibly causing a receptor or a ligand to flow such that the receptor or the ligand flows across each of the porous adsorptive regions of the biochemical analysis unit, the receptor or the ligand being thus subjected to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
b) detecting the receptor or the ligand, which has thus been specifically bound to at least one of the ligands or at least one of the receptors, by the utilization of a labeling substance,
a liquid being forcibly caused to flow, such that the liquid flows across each of the porous adsorptive regions of the biochemical analysis unit, during the specific binding detecting process,
wherein a liquid, which has been subjected to gas content decreasing processing for decreasing the content of a dissolved gas, is employed as the liquid, which is forcibly caused to flow.

2. An assay method using a biochemical analysis unit, comprising the steps of:
i) obtaining a biochemical analysis unit provided with a plurality of porous adsorptive regions, to which ligands or receptors have been bound respectively, and
ii) performing a specific binding detecting process comprising the steps of:
a) forcibly causing a receptor or a ligand to flow such that the receptor or the ligand flows across each of the porous adsorptive regions of the biochemical analysis unit, the receptor or the ligand being thus subjected to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
b) detecting the receptor or the ligand, which has thus been specifically bound to at least one of the ligands or at least one of the receptors, by the utilization of a labeling substance,
a liquid being forcibly caused to flow, such that the liquid flows across each of the porous adsorptive regions of the biochemical analysis unit, during the specific binding detecting process,
wherein bubble removing processing for removing bubbles, which are present in the liquid, from the liquid is performed during the flowing of the liquid.

3. An assay method using a biochemical analysis unit, comprising the steps of:
i) obtaining a biochemical analysis unit provided with a plurality of porous adsorptive regions, to which ligands or receptors have been bound respectively, and
ii) performing a specific binding detecting process comprising the steps of:
a) forcibly causing a receptor or a ligand to flow such that the receptor or the ligand flows across each of the porous adsorptive regions of the biochemical analysis unit, the receptor or the ligand being thus subjected to specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
b) detecting the receptor or the ligand, which has thus been specifically bound to at least one of the ligands or at least one of the receptors, by the utilization of a labeling substance,
a liquid being forcibly caused to flow, such that the liquid flows across each of the porous adsorptive regions of the biochemical analysis unit, during the specific binding detecting process,
wherein bubble dissolving processing for dissolving bubbles, which are present in the liquid, is performed during the flowing of the liquid.

4. A method as defined in claim 1 wherein the specific binding detecting process comprises the steps of:
a) forcibly causing a reaction liquid containing a labeled receptor or a labeled ligand, which has been labeled with a labeling substance, to flow such that the reaction liquid flows across each of the porous adsorptive regions of the biochemical analysis unit provided with the plurality of the porous adsorptive regions, to which the ligands or the receptors have been bound respectively, the labeled receptor or the labeled ligand being thus subjected to the specific binding with the ligands or the receptors, each of which has been bound to one of the porous adsorptive regions of the biochemical analysis unit, the labeled receptor or the labeled ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
b) detecting the labeled receptor or the labeled ligand, which has thus been specifically bound to at least one of the ligands or at least one of the receptors, by the utilization of the labeling substance.

5. A method as defined in claim 2 wherein the specific binding detecting process comprises the steps of:
a) forcibly causing a reaction liquid containing a labeled receptor or a labeled ligand, which has been labeled with a labeling substance, to flow such that the reaction liquid flows across each of the porous adsorptive regions of the biochemical analysis unit provided with the plurality of the porous adsorptive regions, to which the ligands or the receptors have been bound respectively, the labeled receptor or the labeled ligand being thus subjected to the specific binding with the ligands or the receptors, each of which has been bound to one of the porous adsorptive regions of the biochemical analysis unit, the labeled receptor or the labeled ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
b) detecting the labeled receptor or the labeled ligand, which has thus been specifically bound to at least one of the ligands or at least one of the receptors, by the utilization of the labeling substance.

6. A method as defined in claim 3 wherein the specific binding detecting process comprises the steps of:
a) forcibly causing a reaction liquid containing a labeled receptor or a labeled ligand, which has been labeled with a labeling substance, to flow such that the reaction liquid flows across each of the porous adsorptive regions of the biochemical analysis unit provided with the plurality of the porous adsorptive regions, to which the ligands or the receptors have been bound respectively, the labeled receptor or the labeled ligand being thus subjected to the specific binding with the ligands or the receptors, each of which has been bound to one of the porous adsorptive regions of the biochemical analysis unit, the labeled receptor or the labeled ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
b) detecting the labeled receptor or the labeled ligand, which has thus been specifically bound to at least one of the ligands or at least one of the receptors, by the utilization of the labeling substance.

7. A method as defined in claim 1 wherein the specific binding detecting process comprises the steps of:
a) subjecting the receptor or the ligand to the specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
b) forcibly causing a reaction liquid containing a labeled body, which has been labeled with a labeling substance, to flow such that the reaction liquid flows across each of the porous adsorptive regions of the biochemical analysis unit, the labeled body being thus specifically bound to the receptor or the ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
c) detecting the receptor or the ligand, which has been specifically bound to at least one of the ligands or at least one of the receptors, by the utilization of the labeled body.

8. A method as defined in claim 2 wherein the specific binding detecting process comprises the steps of:
a) subjecting the receptor or the ligand to the specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
b) forcibly causing a reaction liquid containing a labeled body, which has been labeled with a labeling substance, to flow such that the reaction liquid flows across each of the porous adsorptive regions of the biochemical analysis unit, the labeled body being thus specifically bound to the receptor or the ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
c) detecting the receptor or the ligand, which has been specifically bound to at least one of the ligands or at least one of the receptors, by the utilization of the labeled body.

9. A method as defined in claim 3 wherein the specific binding detecting process comprises the steps of:
a) subjecting the receptor or the ligand to the specific binding with the ligands or the receptors, each of which has been bound to one of the porous adsorptive regions of the biochemical analysis unit, the receptor or the ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
b) forcibly causing a reaction liquid containing a labeled body, which has been labeled with a labeling substance, to flow such that the reaction liquid flows across each of the porous adsorptive regions of the biochemical analysis unit, the labeled body being thus specifically bound to the receptor or the ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
c) detecting the receptor or the ligand, which has been specifically bound to at least one of the ligands or at least one of the receptors, by the utilization of the labeled body.

10. A method as defined in claim 1 wherein the specific binding detecting process comprises the steps of:
a) subjecting an auxiliary substance-bound receptor or an auxiliary substance-bound ligand, to which an auxiliary substance has been bound, to the specific binding with the ligands or the receptors, each of which has been bound to one of the porous adsorptive regions of the biochemical analysis unit, the auxiliary substance-bound receptor or the auxiliary substance-bound ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
b) forcibly causing a reaction liquid containing a labeling substance, which is capable of undergoing specific binding with the auxiliary substance, to flow such that the reaction liquid flows across each of the porous adsorptive regions of the biochemical analysis unit, the labeling substance, which is capable of undergoing specific binding with the auxiliary substance, being thus specifically bound to the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
c) detecting the auxiliary substance-bound receptor or the auxiliary substance-bound ligand, which has been specifically bound to at least one of the ligands or at least one of the receptors, by the utilization of the labeling substance.

11. A method as defined in claim 2 wherein the specific binding detecting process comprises the steps of:
a) subjecting an auxiliary substance-bound receptor or an auxiliary substance-bound ligand, to which an auxiliary substance has been bound, to the specific binding with the ligands or the receptors, each of which has been bound to one of the porous adsorptive regions of the biochemical analysis unit, the auxiliary substance-bound receptor or the auxiliary substance-bound ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
b) forcibly causing a reaction liquid containing a labeling substance, which is capable of undergoing specific binding with the auxiliary substance, to flow such that the reaction liquid flows across each of the porous adsorptive regions of the biochemical analysis unit, the labeling substance, which is capable of undergoing specific binding with the auxiliary substance, being thus specifically bound to the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
c) detecting the auxiliary substance-bound receptor or the auxiliary substance-bound ligand, which has been specifically bound to at least one of the ligands or at least one of the receptors, by the utilization of the labeling substance.

12. A method as defined in claim 3 wherein the specific binding detecting process comprises the steps of:
a) subjecting an auxiliary substance-bound receptor or an auxiliary substance-bound ligand, to which an auxiliary substance has been bound, to the specific binding with the ligands or the receptors, each of which has been bound to one of the porous adsorptive regions of the biochemical analysis unit, the auxiliary substance-bound receptor or the auxiliary substance-bound ligand being thereby specifically bound to at least one of the ligands, each of which has been bound to one of the porous adsorptive regions of the biochemical analysis unit, or at least one of the receptors, each of which has been bound to one of the porous adsorptive regions of the biochemical analysis unit,
b) forcibly causing a reaction liquid containing a labeling substance, which is capable of undergoing specific binding with the auxiliary substance, to flow such that the reaction liquid flows across each of the porous adsorptive regions of the biochemical analysis unit, the labeling substance, which is capable of undergoing specific binding with the auxiliary substance, being thus specifically bound to the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
c) detecting the auxiliary substance-bound receptor or the auxiliary substance-bound ligand, which has been specifically bound to at least one of the ligands or at least one of the receptors, by the utilization of the labeling substance.

13. A biochemical analysis apparatus, comprising:
i) a reaction vessel, which is provided with a support section for releasably supporting a biochemical analysis unit within the reaction vessel, the biochemical analysis unit being provided with a plurality of porous adsorptive regions, to which ligands or receptors have been bound respectively, the reaction vessel being adapted to perform specific binding of a specific binding substance with the ligands or the receptors, each of which has been bound to one of the porous adsorptive regions of the biochemical analysis unit, the specific binding substance being capable of undergoing the specific binding with the ligands or the receptors, and
ii) flowing means for forcibly causing a reaction liquid containing the specific binding substance to flow within the reaction vessel such that the reaction liquid containing the specific binding substance flows across each of the porous adsorptive regions of the biochemical analysis unit,
wherein the apparatus further comprises bubble removing means for performing bubble removing processing for removing bubbles, which are present in the reaction liquid, from the reaction liquid, which is flowing.

14. A biochemical analysis apparatus, comprising:
i) a reaction vessel, which is provided with a support section for releasably supporting a biochemical analysis unit within the reaction vessel, the biochemical analysis unit being provided with a plurality of porous adsorptive regions, to which ligands or receptors have been bound respectively, the reaction vessel being adapted to perform specific binding of a specific binding substance with the ligands or the receptors, each of which has been bound to one of the porous adsorptive regions of the biochemical analysis unit, the specific binding substance being capable of undergoing the specific binding with the ligands or the receptors, and
ii) flowing means for forcibly causing a reaction liquid containing the specific binding substance to flow within the reaction vessel such that the reaction liquid containing the specific binding substance flows across each of the porous adsorptive regions of the biochemical analysis unit,
wherein the apparatus further comprises bubble dissolving means for performing bubble dissolving processing for dissolving bubbles, which are present in the liquid, on the reaction liquid, which is flowing.

15. An apparatus as defined in claim 13 wherein the reaction vessel is adapted to perform specific binding of a labeled receptor or a labeled ligand, which has been labeled with a labeling substance, with the ligands or the receptors, each of which has been bound to one of the porous adsorptive regions of the biochemical analysis unit, and
the flowing means forcibly causes a reaction liquid containing the labeled receptor or the labeled ligand to flow such that the reaction liquid flows across each of the porous adsorptive regions of the biochemical analysis unit.

16. An apparatus as defined in claim 14 wherein the reaction vessel is adapted to perform specific binding of a labeled receptor or a labeled ligand, which has been labeled with a labeling substance, with the ligands or the receptors, each of which has been bound to one of the porous adsorptive regions of the biochemical analysis unit, and
the flowing means forcibly causes a reaction liquid containing the labeled receptor or the labeled ligand to flow such that the reaction liquid flows across each of the porous adsorptive regions of the biochemical analysis unit.

17. An apparatus as defined in claim 13 wherein the reaction vessel is adapted to perform:
a) specific binding of the receptor or the ligand with the ligands or the receptors, each of which has been bound to one of the porous adsorptive regions of the biochemical analysis unit, and
b) specific binding of a labeled body, which has been labeled with a labeling substance, with the receptor or the ligand, which has been specifically bound to at least one of the ligands or at least one of the receptors, and
the flowing means forcibly causes a reaction liquid containing the labeled body to flow such that the reaction liquid flows across each of the porous adsorptive regions of the biochemical analysis unit.

18. An apparatus as defined in claim 14 wherein the reaction vessel is adapted to perform:
a) specific binding of the receptor or the ligand with the ligands or the receptors, each of which has been bound to one of the porous adsorptive regions of the biochemical analysis unit, and
b) specific binding of a labeled body, which has been labeled with a labeling substance, with the receptor or the ligand, which has been specifically bound to at least one of the ligands or at least one of the receptors, and
the flowing means forcibly causes a reaction liquid containing the labeled body to flow such that the reaction liquid flows across each of the porous adsorptive regions of the biochemical analysis unit.

19. An apparatus as defined in claim 13 wherein the reaction vessel is adapted to perform:
a) specific binding of an auxiliary substance-bound receptor or an auxiliary substance-bound ligand, to which an auxiliary substance has been bound, with the ligands or the receptors, each of which has been bound to one of the porous adsorptive regions of the biochemical analysis unit, and
b) specific binding of a labeling substance, which is capable of undergoing specific binding with the auxiliary substance, with the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
the flowing means forcibly causes a reaction liquid containing the labeling substance, which is capable of undergoing the specific binding with the auxiliary substance, to flow such that the reaction liquid flows across each of the porous adsorptive regions of the biochemical analysis unit.

20. An apparatus as defined in claim 14 wherein the reaction vessel is adapted to perform:
a) specific binding of an auxiliary substance-bound receptor or an auxiliary substance-bound ligand, to which an auxiliary substance has been bound, with the ligands or the receptors, each of which has been bound to one of the porous adsorptive regions of the biochemical analysis unit, and
b) specific binding of a labeling substance, which is capable of undergoing specific binding with the auxiliary substance, with the auxiliary substance-bound receptor or the auxiliary substance-bound ligand having been specifically bound to at least one of the ligands, each of which has been bound to one of the porous adsorptive regions of the biochemical analysis unit, or at least one of the receptors, each of which has been bound to one of the porous adsorptive regions of the biochemical analysis unit, and
the flowing means forcibly causes a reaction liquid containing the labeling substance, which is capable of undergoing the specific binding with the auxiliary substance, to flow such that the reaction liquid flows across each of the porous adsorptive regions of the biochemical analysis unit.
